(19) United States
(12) Patent Application Publication
(10) Pub. No.: US 2014/0243712 A1
(43) Pub. Date: Aug. 28, 2014

(54) THROMBOLYSIS IN RETINAL VESSELS WITH ULTRASOUND

(71) Applicants: Mark Humayun, Glendale, CA (US); Xiaochen Xu, Los Angeles, CA (US); Qifa Zhou, Arcadia, CA (US); K. Kirk Shung, Monterey Park, CA (US); Hossein Ameri, Alhamra, CA (US); Gerald Chader, Pasadena, CA (US)

(72) Inventors: Mark Humayun, Glendale, CA (US); Xiaochen Xu, Los Angeles, CA (US); Qifa Zhou, Arcadia, CA (US); K. Kirk Shung, Monterey Park, CA (US); Hossein Ameri, Alhamra, CA (US); Gerald Chader, Pasadena, CA (US)

(73) Assignee: Doheny Eye Institute, Los Angeles, CA (US)

(21) Appl. No.: 13/780,535

(22) Filed: Feb. 28, 2013

(51) Int. Cl. A61N 7/00

(52) U.S. Cl. CPC A61N 7/00
USPC 601/2

(57) ABSTRACT

Systems and methods are described providing for the use of ultrasound energy to effect the dislodging of one or more blood clots inside blood vessels. Such clots can include those inside retinal vessels, especially in patients with central retinal vein occlusion. Embodiments of the present disclosure may be used for any retinal arterial or venous occlusion. In exemplary embodiments, a small probe can be inserted into the eye of a patient and placed over the retinal vessels. Acoustic streaming created by the probe can be directed to an area or region including targeted blood vessels, resulting in increased flow in one or more retinal veins and facilitating or effecting mechanical dislodging of one or more blood clots in the targets blood vessels. Exemplary embodiments can utilize ultrasonic energy produced at a frequency of approximately 44 MHz to 46 MHz with pulse repetition frequencies of approximately 100 Hz to 100 kHz.

### RELATED APPLICATIONS

This application claims the benefit of U.S. Provisional Patent Application No. 60/909,522 filed 2 Apr. 2007, the entire content of which application is incorporated herein by reference. This application is also related to U.S. Provisional Patent Application No. 60/909,496 filed 2 Apr. 2007 and U.S. patent application Ser. No. ______ entitled “Preoperative and Intra-Operative Lens Hardness Measurement by Ultrasound” filed 2 Apr. 2008; and also U.S. Provisional Patent Applications No. 60/911,385 filed 12 Apr. 2007, and No. 61/030,075 filed 20 Feb. 2008, the entire contents of all of which applications are incorporated herein by reference.

### BACKGROUND

The occlusion or blockage of blood vessels, for example, within the eye can produce major health problems, such as loss of vision. An example is central retinal artery occlusion (“CRAO”), which is commonly defined as the acute loss of vision in one eye secondary to thrombosis of the central retinal artery.

Prior art blood clot removal strategies include enzymatic and/or mechanical approaches. Clot dissolving, or so-called “clot busting,” drugs (e.g., tissue plasminogen activator, or “tPA”), can be used to relieve the obstruction to blood flow. For such clot dissolving strategies, it has been reported that applying ultrasound on clotted vessels can help dissolve blood clots further. The frequency range of the ultrasound as used for such use has been below 1 MHz. Low intensity ultrasound has been used as a technique to accelerate clot dissolving. Methods of improving enzymatic thrombolysis with ultrasound include intra-arterial delivery of thrombolytic agents with an ultrasound-emitting catheter and targeted and non-targeted non-invasive transcranial ultrasound delivery during intravenous thrombolytic infusion.

Mechanical thrombolysis with ultrasound in prior art techniques has typically required the use of high intensities of acoustic power at the clot (>2 W/cm²). Due to the high intensity ultrasound, unwanted side effects have often resulted and these can include tissue thermal and mechanical injury. Use of a micro-air bubble based contrast agent, which is exposed under ultrasound, has been demonstrated to be a noninvasive, nonlytic approach for clot dissolution.

A pulsed-wave Doppler system with a PMN-PT needle transducer has been developed to measure the blood flow velocity in selected retinal vessels. See, e.g., Emanuel J. Gottlieb, et al., “PMN-PT High Frequency Ultrasonic Needle Transducers for Pulsed Wave Doppler In The Eye,” 2005 IEEE Ultrasonics Symposium (IEEE 2005), the contents of which are incorporated herein by reference in their entirety. Ultrasonic techniques have also been utilized in surgical procedures on the eye for imaging structure and/or tissue of a surgical site. See, e.g., U.S. Pat. No. 6,676,607 to de Juan, Jr. et al., the contents of which are incorporated herein by reference in their entirety.

While prior art techniques have proven useful for their respective intended purposes, they can present difficulties or limitations with respect to thrombolysis in retinal eye vessels. Such drawbacks have included the unwanted side effects on human tissue from high power intensities.

### SUMMARY OF THE DISCLOSURE

Systems and methods according to the present disclosure provide for the use of ultrasound energy to effect the dislodging of one or more blood clots inside blood vessels anywhere in the body. Such blood vessels can be retinal vessels, especially in patients with central retinal vein occlusion. Embodiments of the present disclosure may be used for any retinal arterial or venous occlusion.

In exemplary embodiments, a small probe can be inserted into the eye of a patient and placed over the retinal vessels. Acoustic streaming created by the probe can be directed to an area/regions including targeted blood vessels, resulting in increased flow in one or more retinal veins and helping to or effecting mechanical dislodging of a blood clot. In exemplary embodiments, the probe can be a needle probe having a piezoelectric transducer that is configured and arranged to operate at high ultrasonic frequencies, e.g., between about 40 MHz to about 50 MHz, with exemplary embodiments operational at about 44 MHz to about 45 MHz. The tip of the probe can be angled as desired, e.g., with a desired angle (0, 30, 45, 60, etc.) between a face or surface of the tip and the longitudinal or long axis of the probe.

Further embodiments of the present disclosure can include or be directed to ultrasonic signal generation and/or detection systems that can function to supply a probe (e.g., one suitable for insertion into an eye) with ultrasonic energy. Exemplary embodiments can utilize pulsed wave Doppler techniques and be based on coherent demodulation and sample-and-hold techniques. In exemplary embodiments, a system can include a needle transducer, a pulser/receiver board including an oscillator operating at an ultrasonic frequency (e.g., 44 MHz or 45 MHz, etc.), a timing circuit, a power amplifier, wide-band low-noise amplifiers, a demodulator, sample-and-hold circuits, and, if desired, audio amplification, which can be implemented with an A/D converter (sound card) and a personal computer.

Exemplary embodiments of methods or processes according to the present disclosure can include inserting an ultrasound transducer into a patient's eye, where the transducer can be placed or located over retinal blood vessels of the eye. Ultrasonic energy emanating from the transducer can be directed to the retinal vessels for effecting thrombolysis in one or more blood vessels.

Aspects of the present disclosure can provide one or more of the following, as advantages over existing technology: (i) increased lateral resolution, as high frequency probes can derive or produce better lateral resolution than low frequency probes; this can allow an acoustic beam to be focused in a limited area; (ii) use of a high frequency small probe makes it possible to deliver the ultrasound energy to the selected retinal vessels, which are usually under 200 μm in diameter, from a close distance; (iii) use of acoustic streaming, as opposed to shockwaves, can reduce the risk of collateral damage to surrounding nerve fiber layers; and/or (iv) relatively inexpensiveness for systems/components according to the present disclosure, including those offering quantitative flow velocity for measuring and blood clot dislodging capabilities.

Other features and advantages of the present disclosure will be understood upon reading and understanding the detailed description of exemplary embodiments, described herein, in conjunction with reference to the drawings.

### BRIEF DESCRIPTION OF DRAWINGS

Aspects of the disclosure may be more fully understood from the following description when read together with the accompanying drawings, which are to be regarded as illustrative in nature, and not as limiting. The drawings are not necessarily to scale, emphasis instead being placed on the principles of the disclosure. In the drawings:

FIG. 1 depicts a design cross section of a suitable PMN-PT needle transducer for thrombolysis, in accordance with an embodiment of the present disclosure;

FIG. 2A is a perspective view of a PMN-PT needle transducer in accordance with an exemplary embodiment of the present disclosure; FIG. 2B includes a perspective view of embodiments of needle transducers in accordance with the present disclosure;

FIG. 3 is a box diagram representing a system in accordance with an embodiment of the present disclosure; and

FIG. 4 depicts a method according to an exemplary embodiment of the present disclosure.

One skilled in the art will appreciate that the embodiments depicted in the drawings are illustrative and that variations of those shown, as well as other embodiments described herein, may be envisioned and practiced within the scope of the present disclosure.

### DETAILED DESCRIPTION

Systems and methods according to the present disclosure provide for the use of ultrasound energy to effect thrombolysis, or the dislodging of blood clots, inside blood vessels anywhere in the body. For such techniques, ultrasonic transducers, e.g., needle probes, may be employed. Such techniques may be especially useful for thrombolysis on retinal blood vessels in patients with central retinal vein occlusion, though embodiments of the present disclosure may be used for any retinal arterial or venous occlusion. Ultrasonic transducers or needle probes as disclosed herein can be combined with various endoscopes used throughout body cavities, e.g., as used to evaluate tumors such as melanoma, etc. Ultrasonic transducers or needle probes according to the present disclosure may also be combined within or employed with cryogenic (cryo), laser, illumination, and/or cautery probes used for various parts of body, including internal body cavities.

In exemplary embodiments, a small probe can be inserted into the eye of a patient and placed over the retinal vessels. Acoustic streaming created by the probe can be directed to an area/regions including targeted blood vessels, resulting in increased flow in one or more retinal veins and helping to or effecting mechanical dislodging of a blood clot. In exemplary embodiments, the probe can be a needle probe having a piezoelectric transducer that is configured and arranged to operate at high ultrasonic frequencies, e.g., between about 1 MHz to about 50 MHz, with exemplary embodiments operational at about 44 MHz to about 46 MHz, e.g., 45 MHz. Other ranges of ultrasonic operation include from about 1.0 MHz to about 60 MHz or beyond. The tip of the probe can be angled as desired, e.g., with a desired angle (0, 30, 45, 60, etc.) between a face or surface of the tip and the longitudinal or long axis of the probe.

Further embodiments of the present disclosure can include or be directed to ultrasonic signal generation and/or detection systems that can function to supply a probe (e.g., one suitable for insertion into an eye) with ultrasonic energy. Exemplary embodiments can utilize pulsed wave Doppler techniques and be based on coherent demodulation and sample-and-hold techniques. In exemplary embodiments, a system can include a needle transducer, a pulser/receiver board including an oscillator operating at an ultrasonic frequency (e.g., 44 MHz, 45 MHz, or 46 MHz, etc.), a timing circuit, a power amplifier, wide-band low-noise amplifiers, a demodulator, sample-and-hold circuits, and, if desired, audio amplification, which can be implemented with an A/D converter (sound card) and a personal computer.

Exemplary embodiments of methods or processes according to the present disclosure can include inserting an ultrasound transducer into a patient's eye, where the transducer can be placed or located over retinal blood vessels of the eye. Ultrasonic energy emanating from the transducer can be utilized to produce acoustic streaming—a term referring to a bulk fluid flow resulting from an acoustic field propagating in a fluid medium—to effect thrombolysis in one or more targeted blood vessels, e.g., in a central retinal artery. For some applications/embodiments, the flow velocity introduced by acoustic streaming can be as high as 14 cm/s, or more (typical blood velocities in human retinal veins are around 5 cm/s). The acoustic streaming produced can be used for thrombolysis to remove or mitigate blood clots of blood vessels. The acoustic streaming may be used to accelerate the blood flow in retinal veins significantly, and the blood clot may be dislodged and/or removed. In exemplary embodiments, such techniques can be utilized in or near patient's eye (or the eye of an animal).

Systems according to the present disclosure can also be used to excite a probe to create acoustic streaming in selected blood vessels. In vitro and in vivo experiments by the present inventors have shown that significant acoustic streaming can be created by embodiments of the present disclosure to move a small blood clot and effect thrombolysis.

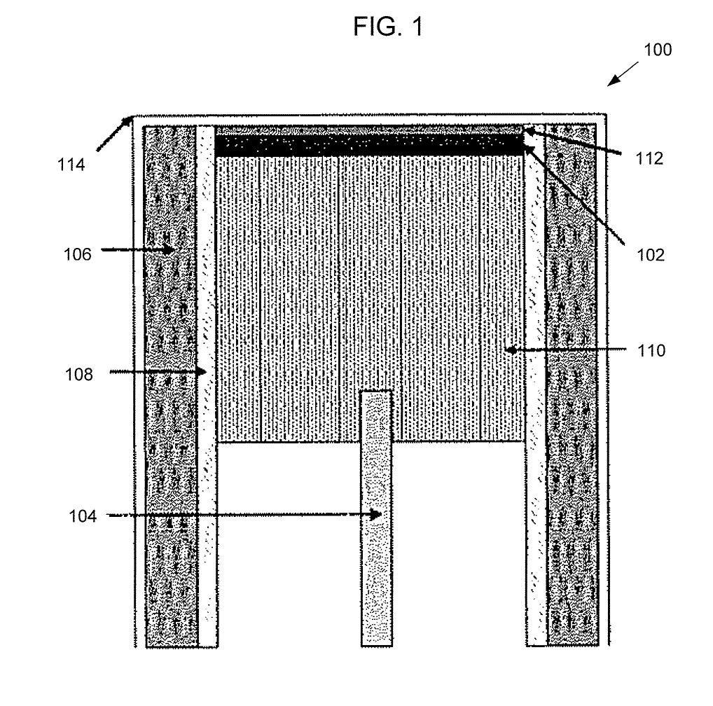

FIG. 1 depicts a design cross section of an exemplary needle transducer or probe 100 for thrombolysis, in accordance with exemplary embodiments of the present disclosure.

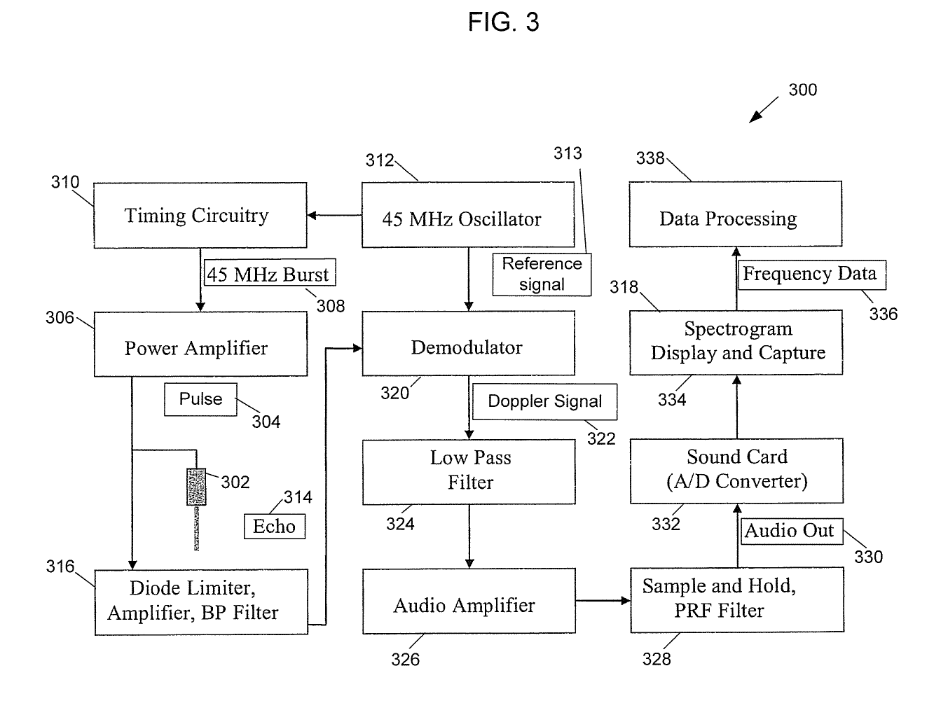

As shown in FIG. 1, the probe 100 can include a piezoelectric material 102 disposed with a needle housing 106. The piezoelectric material 102 can be any suitable active piezoelectric material. One suitable piezoelectric material is lead magnesium niobate lead titanate (e.g., PNM-33% PT). The piezoelectric material may be attached (directly or indirectly, and with suitable electrical configuration/connection) to an electrical connector 104 by suitable fabrication/construction techniques. For example, Cr/Au electrodes can be used to connect the piezoelectric material 102 to the electrical connector 104, though other conductive material(s) may be used. Housing 106 can be of a desired diameter and material, e.g., steel of 1 mm diameter, which size can be suitable (or selected) for insertion into an ocular incision. The needle housing 106 can surround a tube 108 of electrically insulating/isolating material, e.g., made of polyimide fabricated by suitable techniques. The electrical connector may be one suitable for connection to a control system configured to control the production of acoustic energy from the transducer, for example system 300 show and described for FIG. 3 herein.

Continuing with the description of probe 100, a conductive backing material 110 can be located between the piezoelectric material 102 and the electrical connector 104. A matching layer 112 may be located on or adjacent to the side of the probe from which acoustic energy is to be produced. A protective coating 114 may optionally be present, with parylene being an exemplary material for the protective coating, though others may be used.

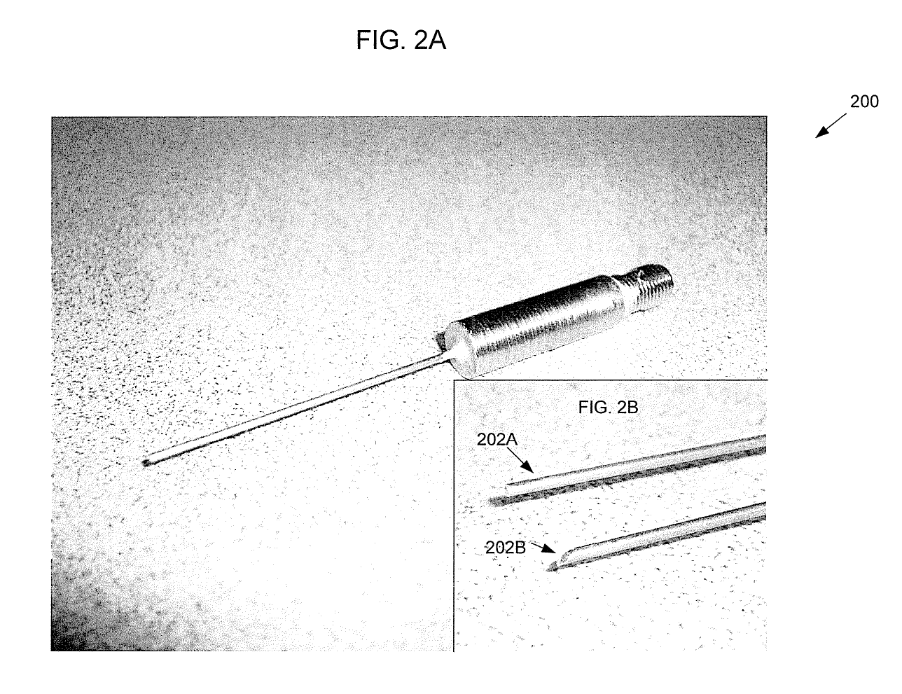

FIG. 2A is a perspective view of an exemplary PMN-PT needle transducer 200. FIG. 2B is an inset showing embodiments of the needle transducer tip having either a 0° or 45° tip (202A, 202B) in accordance with an embodiments of a system according to the present disclosure. Other angles may be used for the tip configuration.

For the exemplary embodiment of needle transducer 200 in FIG. 2A, a 700 μm thick PMN-PT (HC Material Corp., Urbana, Ill.) was lapped to 51 μm. A matching layer made of Insulcast 501 and Insulcure 9 (American Safety Technologies, Roseland, N.J.) and 2-3 μm silver particles (Sigma-Aldrich Inc., St. Louis, Mo.) was cured over the PMN-PT and lapped to 10 μm. A conductive backing material, E-solder 3022 (VonRoll Isola, New Haven, Conn.), was cured over the opposite side of the PMN-PT and lapped to under 3 mm. Active element plugs were diced out at 0.4 mm aperture (0.4 mm×0.4 mm) and housed using Epotek 301 (Epoxy Technology Inc., Billerica, Mass.) within a polyimide tube with inner diameter of 0.57 mm (MedSource Technologies, Trenton, Ga.). An electrical connector was fixed to the conductive backing using a conductive epoxy. The polyimide tube provided electrical isolation from the 20 gage needle housing with inner diameter 0.66 mm. An electrode was sputtered across the silver matching layer and the needle housing to form the ground plane connection. Vapor deposited parylene with thickness of 13 μm was used to coat the aperture and the needle housing.

A needle probe according to the present disclosure, such as depicted in FIGS. 1-2, can provide the advantages of high efficiency, affordable price, and simple fabrication procedures. Such a probe can have a (natural) focal point at a desired distance from the tip of the prove, e.g., at approximately 1˜2 mm. For an exemplary embodiment, a PMN-NT probe according to FIGS. 1-2 had a measured lateral resolution of about 300 μm at a distance of 2 mm. Such lateral resolution and focal distance parameters can be particularly useful for clot dislodging as a typical central retinal vein locates at 1 mm below the optical nerve.

As described previously, a suitable electronic system can be used to control/excite a needle probe (e.g., probe 200 of FIG. 2A) used for ultrasound-based thrombolysis according to the present disclosure.

FIG. 3 is a box diagram representing an exemplary system 300 (or controller) for controlling a needle probe (e.g., a PMN-PT needle probe described for FIGS. 1-2), in accordance with an embodiment of the present disclosure. System 300 can include both (i) excitation components for controlling the ultrasonic output of a transducer, e.g., needle probes 100 and 200 of FIGS. 1-2, and also (ii) optional circuitry/components for Doppler detection of blood flow in retinal blood vessels.

As shown in FIG. 3, system 300 can include a piezoelectric transducer or probe 302. Probe 302 can be connected to, or operation to receive signals/pulses from a pulse generation block, which can include a power amplifier 306, timing circuitry 310, and a suitable clock or oscillator 312, e.g., a 45 MHz clock generator (or oscillator). System 300 can operate as a pulser, e.g., a N-cycle bipolar pulser, to generate one or more suitable pulses for supplying the transducer 302 with electrical energy for conversion to acoustic ultrasound energy. In exemplary embodiments, system 300 can produce a N-cycle bi-polar pulse with 70 Vpp, for the control of the associated ultrasonic probe/transducer 302. The pulse repetition frequency (PRF) of the produced pulse(s) produced by system 300 can be adjusted as desired, e.g., from 100 Hz to 100 KHz, and the cycle count of the pulse can be adjusted as desired, e.g., from 1 to 255. Both the PRF and cycle count can correspond to different acoustic intensities (e.g., different flow velocities created by the acoustic streaming).

In addition to pulse generation circuitry/components, system 300 can also include optional Doppler detection circuitry/components for detecting and displaying blood velocity of the retinal vessels. For example, as shown in FIG. 3, system 300 can include the following components/functionality in a suitable configuration: a diode limiter and/or bandpass filter component/block 316; a demodulator 320, which may be configured to receive a reference signal 313 from clock/oscillator 312 and also to produce a Doppler signal 322 indicative of fluid movement. A low pass filter 324 may be connected to the demodulator 320 as shown, passing the Doppler signal 322 to an audio amplifier 326.

Continuing with the description of FIG. 3, system 300 can include a sample and hold (PRF Filter) 328 connected to the audio amplifier 326 and capable of producing an audio output 330. PRF filter 328 can be connected and pass the audio output 330 to a sound card including an A/D converter 332. A spectrogram block 334, e.g., for display and capture information/data can be connected to the sound card 332 and data processing components/circuitry, e.g., for frequency data 336, received from the spectrogram block 334. Other suitable components may be utilized in conjunction with or substitution for the ones shown in FIG. 3.

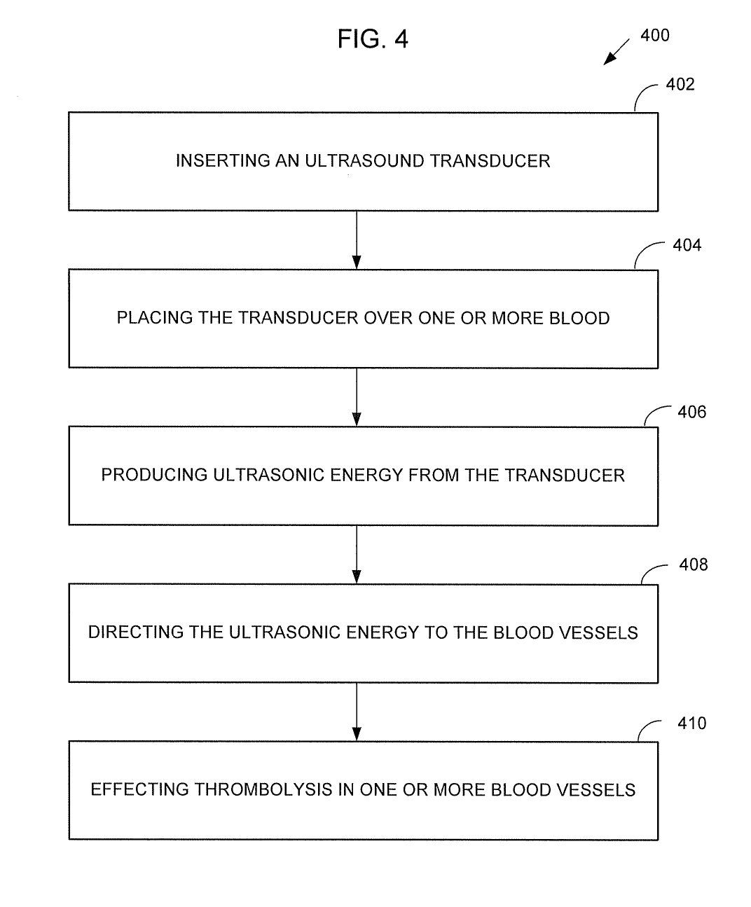

FIG. 4 depicts a method 400 according to an exemplary embodiment of the present disclosure. An ultrasound transducer can be inserted into a patient, as described at 402. In exemplary embodiments, an ultrasound transducer can be inserted into the eye of the patient, though the probe may be inserted into other tissue or bone as well. The transducer may be placed over or adjacent to targeted blood vessels, as described at 404. In exemplary embodiments, the probe/transducer may be placed over or adjacent to retinal blood vessels of the eye. The targeted blood vessels may include one or more blood clots. Ultrasonic energy can be produced from the transducer, as described at 406. For example, an electronic control system according (or similar) to FIG. 3 can be used to control the production, e.g., 406, or ultrasonic energy. The ultrasonic energy may be produced at a desired frequency, e.g., over a range of about 1.0 to about 60 MHz. Exemplary embodiments can utilize ultrasonic energy within a range of about 44 MHz to about 46 MHz, e.g., 45 MHz.

Continuing with the description of method 400, the ultrasonic energy can be directed to the targeted retinal vessels, including those containing blood clots, as described at 408. Directing ultrasonic energy can include producing acoustic streaming in the blood of the targeted blood vessels and/or fluid within the eye itself, e.g., vitreous humor. As described at 410, thrombolysis can accordingly be effected.

In an exemplary embodiment according to the present disclosure, including a control system with the PMN-PT probe, a micro flow phantom blood vessel consisting of a 127˜574 μm tube was constructed for testing purposes. The material of the tube was selected to be similar to real human vessels. Preferably materials used for such a tube are so-called bio-safe materials. Blood was introduced to the tube and clots were allowed to form in the tube. Initial experiments showed that the system with the PMN-PT probe was able to move a blood clot with diameter of 1 mm. Significantly, turbulence caused by the acoustic streaming was observed in the experiments, indicating that the system was suitable for use in dislodging retinal blood clots.

Accordingly, compared to the existing technologies, embodiments of the present disclosure can provide the advantage of instant clot dislodging in less invasive procedures. The effect of clot dislodging can be evaluated by the combined the Doppler system right after the dislodging procedure. During the initial experiments, no significant temperature increasing which may be a major side effect of this technology, was noticed. Cost benefits may also be realized. For example, the total cost of an embodiment of a reusable system according to the present disclosure can be less than $2000.

Moreover, aspects of the present disclosure can provide one or more of the following, as advantages over existing technology: (i) increased lateral resolution, as high frequency probes can derive or produce better lateral resolution than low frequency probes; this can allow an acoustic beam to be focused in a limited area; (ii) use of a high frequency small probe makes it possible to deliver the ultrasound energy to the selected retinal vessels, which are usually under 200 μm in diameter, from a close distance; (iii) use of acoustic streaming, as opposed to shockwaves, can reduce the risk of collateral damage to surrounding nerve fiber layers; and/or (iv) relatively inexpensiveness for systems/components according to the present disclosure, including those offering quantitative flow velocity for measuring and blood clot dislodging capabilities.

While certain embodiments have been described herein, it will be understood by one skilled in the art that the methods, systems, and apparatus of the present disclosure may be embodied in other specific forms without departing from the spirit thereof. For example, while certain piezoelectric materials have been mentioned specifically, others may be used within the scope of the present disclosure. For further example, while embodiments of the present disclosure have been described in the context of the eye, clots may be dislodged and thrombolysis effected in blood vessels in other tissues, regions, and/or organs.

Accordingly, the embodiments described herein are to be considered in all respects as illustrative of the present disclosure and not restrictive.

1. An ultrasonic needle transducer system comprising:
an ultrasonic needle transducer for producing an output of ultrasound energy, the transducer including a piezoelectric material and being configured and arranged for intraocular insertion,
a control unit connected to the ultrasound transducer and configured and arranged to control the production of ultrasound energy from the transducer.

2.-4. (canceled)

5. The system of claim 1, wherein the piezoelectric material comprises PMN-PT.

6.-8. (canceled)

9. The system of claim 1, further comprising a tube of electrically insulating material disposed within the cylindrical needle housing.

10. The system of claim 9, wherein the flexible tube comprises polyimide.

11. The system of claim 5 wherein the PMN-PT comprises PMN-33% PT.

12. The system of claim 1, wherein the control unit comprises timing circuitry and a power amplifier for supplying the transducer with a signal for driving the transducer at a ultrasonic frequency.

13. The system of claim 1, wherein the control unit is configured and arranged to control the intensity of the ultrasonic output of the transducer.

14. The system of claim 1, wherein the control unit is configured and arranged to control the pulse repetition frequency (PRF) of the output of the transducer.

15. (canceled)

16. The system of claim 1, further comprising a spectrogram configured and arranged to display and capture velocity information received from the Doppler processing circuitry.

17. The system of claim 1, wherein the transducer and controller are configured and arranged to produce ultrasonic energy at a frequency of about 1 MHz to about 50 MHz.

18. The system of claim 1, wherein the controller is configured and arranged to produce a pulse repetition frequency of about 100 Hz to about 100 kHz.

19. The system of claim 1, wherein the controller is configured ad arranged to produce a pulse cycle count from 1 to 255.

20. A method of performing thrombolysis in a blood vessel, the method comprising:
inserting the ultrasound transducer into a patient;
placing the transducer over or adjacent to blood vessels of the patient;
producing ultrasonic energy from the transducer;
directing the ultrasonic energy to the retinal vessels; and
effecting thrombolysis in one or more blood vessels.

21.-23. (canceled)

24. The method of claim 20, wherein producing ultrasonic energy from the transducer comprises producing ultrasonic energy at a frequency of about 1 MHz to about 50 MHz.

25. The method of claim 24, wherein the ultrasonic energy is produced at a frequency of about 44 MHz to about 24 MHz.

26. The method of claim 20, wherein producing ultrasonic energy from the transducer comprises producing a pulse repetition frequency of about 100 Hz to about 100 kHz.

27. The method of claim 20, wherein producing ultrasonic energy from the transducer comprises producing a pulse cycle count from 1 to 255.

28.-30. (canceled)

31. The method of claim 20, wherein placing the transducer over or adjacent to blood vessels of the patient comprises placing the transducer over or adjacent to retinal vessels of the eye or the optic nerve of the patient.
